(19) United States
(12) Patent Application Publication
(10) Pub. No.: US 2006/0173344 A1
(43) Pub. Date: Aug. 3, 2006

(54) Method for using a refrigeration system to remove waste heat from an ultrasound transducer

(75) Inventors: Vaughn R. Marian, Saratoga, CA (US); William J. Park, San Jose, CA (US); Timothy E. Petersen, Mountain View, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC.

(21) Appl. No.: 11/039,588

(22) Filed: Jan. 19, 2005

(51) Int. Cl. A61B 8/14

(52) U.S. Cl. 600/459

(57) ABSTRACT

Methods and systems are provided for cooling an ultrasound transducer using a refrigeration system located within the imaging system. A closed loop of recirculating coolant located in the transducer assembly transports waste heat from the remotely located heat producing acoustic components or active electronics components to a thermally conductive shoe, located within the transducer connector. Thermally conductive materials in each connector, the ultrasound system connector and the transducer assembly connector, are positioned in contact to thermally conduct heat from the transducer assembly to a refrigeration system, located in the imaging system, free of fluid transfer.

### BACKGROUND

The present invention relates to diagnostic ultrasound transducer cooling. Medical diagnostic ultrasound piezoelectric devices and supporting electronics generate significant waste heat during operation. Generally, transducers that can be operated at higher power levels are favored. Such transducers provide superior diagnostic performance due to increased transmit energy into the body. Integration of heat generating low noise amplifiers in close proximity to the acoustic receivers increases the signal-to-noise performance for the detected ultrasonic energy.

There are regulatory limits on the temperatures that are allowed for the surfaces of the transducer. For example, the regulatory limit for the surface of a diagnostic ultrasound transducer that is in contact with the patient is 43 degrees C.

In general, waste heat generated in the transducer is dissipated by passive methods to either the patient or to the atmosphere. Because of the limited surface area of a practical ultrasound transducer, there are limitations on the amount of heat that can be transferred into the environment and the patient by conduction, by radiation and by free convection from temperature compliant surfaces. The practical limits for energy dissipation for small diagnostic ultrasound transducers on the order of 1 to 2 watts, steady state.

In U.S. Pat. No. 5,560,362, active cooling increases the amount of heat that can be removed from a transducer. In general, active cooling schemes use coolant, flowing in a closed loop system to transfer waste heat to a location where it can be efficiently dissipated into the atmosphere. Fans and fluid/air heat exchangers within the transducer assembly system connector facilitate dissipation of waste heat to the environment. There are practical limits to how much heat can be dissipated in this manner, due to the limited volume for the heat exchanger and fan, and due to the relatively small temperature difference between the coolant and the atmosphere. Practical limits may be on the order of 5-12 watts, steady state.

In another approach, the heat dissipation hardware is located within the system connector or the imaging system rather than the transducer assembly connector. Fluid is conveyed from the connector to the imaging system. Given the detachable connection of the transducer assembly to the system connector, a practical method for conveying fluid to and from the system may be a challenge.

### BRIEF SUMMARY

By way of introduction, the preferred embodiments described below include methods and systems for cooling an ultrasound transducer using a refrigeration active cooling system. Because of the size of the imaging system, it may be more practical to place the refrigeration system in the ultrasound system or consol. However, a bi-directional fluid transfer between the imaging system and the transducer assembly may be avoided. A cooling system, using a closed loop of coolant, is located within the transducer assembly for extracting waste heat from the acoustic components and/or supporting electronics and conveying the heat to a thermal interface between the transducer assembly connector and the ultrasound imaging system. Thermally conductive components in each connector, the ultrasound system connector and the transducer assembly connector, are positioned in contact to thermally conduct heat from the transducer assembly to the refrigeration system, free of fluid transfer.

In a first aspect, a system is provided for cooling an ultrasound transducer. An ultrasound transducer assembly is operable to releasably connect with an ultrasound imaging system. A refrigeration cooling device is within the ultrasound system. A connector is operable to thermally conduct between the ultrasound transducer assembly and the refrigeration cooling device free of fluid transfer.

In a second aspect, a system is provided for cooling an ultrasound transducer. An ultrasound transducer assembly has a first fluid path extending from adjacent to a transducer array to a first thermally conductive shoe in the first connector. An ultrasound system has a refrigeration cooling device and a second connector operable to connect with the first connector and has a second thermally conductive shoe in the second connector. The second thermally conductive shoe contacts the first thermally conductive shoe if the ultrasound transducer assembly is connected with the ultrasound system. The refrigeration cooling device thermally connects with the second thermally conductive shoe.

In a third aspect, a method is provided for cooling an ultrasound transducer. Active cooling is provided within an ultrasound system. Heat is conducted from the ultrasound transducer in response to the active cooling within the ultrasound system free of fluid connection between the ultrasound transducer and the ultrasound system.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a diagram of a first embodiment of an active cooling system for an ultrasound transducer;

FIG. 2 is a cross-section diagram of one embodiment of a transducer assembly cable for a fluid based active cooling system;

FIG. 3 is a diagram of a second embodiment of an active cooling system for an ultrasound transducer;

FIG. 4 is a graphical representation of one embodiment of a heat flow diagram;

FIG. 5 is a diagram of a third embodiment of an active cooling system for an ultrasound transducer;

FIG. 6 is a diagram of a fourth embodiment of an active cooling system for an ultrasound transducer;

FIG. 7 is a diagram of a fifth embodiment of an active cooling system for an ultrasound transducer; and

FIG. 8 is a diagram of a sixth embodiment of an active cooling system for an ultrasound transducer.

### DETAILED DESCRIPTION OF THE DRAWINGS AND PRESENTLY PREFERRED EMBODIMENTS

Regulations require that ultrasound transducers, used for medical diagnostic procedures, be limited to no more than 43 degrees C. where the transducer touches the patient. With a presumed ambient air temperature of 25 degrees C., only 18 degrees C. temperature difference facilitates heat removal by passive methods that include natural convection, conduction, and radiation.

To provide additional heat removal, a cooling system within the transducer assembly, utilizing a recirculating liquid coolant, is exploited to transport waste heat from the heat generating acoustic or electronics components within transducer, along its cable, to the transducer connector. Instead of attempting to dissipate the waste heat in the relatively small connector, the heat is transferred to the ultrasound imaging system by thermal conduction. Once within the imaging system, the waste heat is dissipated into the atmosphere with the aid of a vapor/liquid or some other kind of refrigeration system. Because of the ability of a refrigeration system to pump heat up a temperature gradient, the thermal receptacle in the system can be maintained at a temperature far below ambient air temperature. This enables the temperature difference between the heat generating transducer and the heat sink, now located in the imaging system, to be increased to a much higher value, such as 40 to 60 degrees C. The increased temperature difference is exploited to increase the amount of waste heat that can be removed from the remotely located transducer. More heat can be generated within the transducer before regulatory surface temperature limits are exceeded.

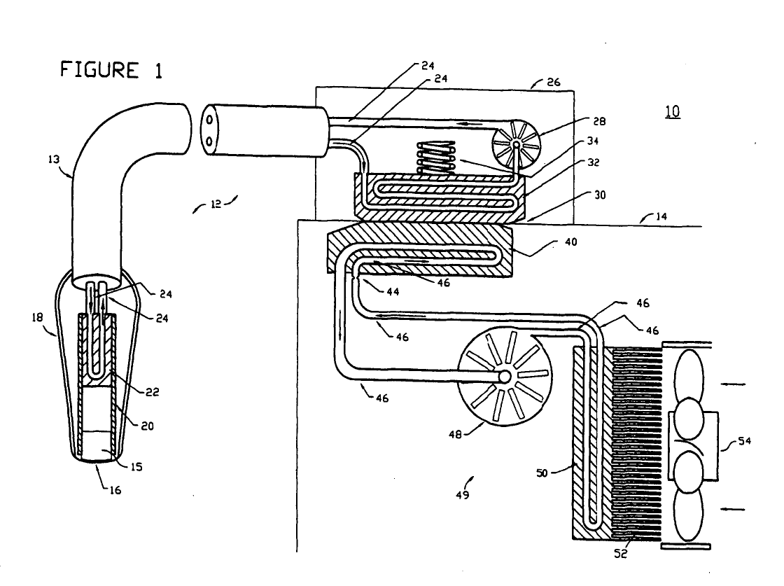

FIG. 1 shows a system 10 for cooling a component or components of an ultrasound transducer assembly 12 by use of a vapor/liquid refrigeration system located within the ultrasound imaging system 14. An ultrasound system 14 incorporates components (40-54) of a refrigeration system for enhanced heat removal. Waste heat is generated by the transducer acoustic components 15 and/or by supporting electronics, not illustrated, located within the transducer housing 18 or the connector 26 during normal operation. Some of this waste heat is transferred to the ultrasound system 14 and dissipated to the atmosphere. Any range of temperature gradients may be provided, such as 20-60 degrees C. from an acoustic window 16 to the refrigeration system located within the ultrasound system 14. Temperatures discussed herein as an example of the temperature gradient associated with different components or transfers are provided for a functional description only and have not been calculated. To simplify functional descriptions, steady state operation is presumed.

The ultrasound transducer assembly 12 is comprised of the transducer housing 18, and all components within, a cable assembly 13, and a connector 26 and all the components within. Ultrasound energy, generated in the acoustic stack 15, travels through the acoustic window 16 to the patient, not illustrated. Small amounts of ultrasound energy, reflected from anatomical features within the patient return to the acoustic stack 15 where they are converted into small electrical signals that are either processed by electrical components located within the housing 18, or directly conducted to the imaging system for conversion to a clinically useful diagnostic image. The active cooling system components within the transducer housing 18 include a thermal plate 20, a heat exchanger 22, and a fluid path 24. The active cooling system components located within the connector include a thermally conductive shoe 32 with a mating surface 30, a spring 34, and a re-circulation pump 28. Additional, different or fewer components may be provided for transferring heat from components located within the transducer housing 18 to the connector 26 instead of or in addition to the fluid path 24. As another example, the thermal plate 20, the heat exchanger 22, and/or other components are not provided.

The ultrasound transducer assembly 12 is releasably connectable with the ultrasound system 14. In addition to the components listed above, the connector 26 includes electrical interconnections or metallic contacts for mating with a corresponding connector on the ultrasound system 14. The electrical interconnections provide transmit waveforms from the ultrasound system 14 to the transducer 15 for generating acoustic wave fronts to scan a patient and/or provide received signals from the transducer 15 to the ultrasound system 14 for imaging. In one embodiment, some or all of the electronics used to generate the transmit waveforms are located within the transducer assembly 12, such as being within the transducer housing 18 or both the transducer housing 18 and the connector 26. In a different or additional embodiment, some of the receive electronics, such as a multiplexer, pre-amplifiers or filters, are also positioned in the transducer assembly 12. Alternatively, the transducer assembly 12 is free of active electronics. Mechanical connection is also provided for releasably connecting the connector 26 with the ultrasound system 14. For example, latches, snap fit mating surfaces, threading or other mechanism holds the connector 26 to the ultrasound system 14 during use. In one embodiment, the transducer assembly 12 includes components disclosed in U.S. Pat. No. 5,560,362, the disclosure of which is incorporated herein by reference.

The acoustic ultrasound transducer 15 comprises a one dimensional or multi-dimensional array of elements. The transducer 15 is comprised of a matching layer, backing block, individual piezo-electric or CMUT elements, and a flexible circuit for electrical interconnection. High voltage transmit waveforms are applied to the transducer 15 for generating acoustic wavefronts. The transduction of the transmit waveforms generates heat.

The acoustic window 16 comprises Pebax, epoxy, silicone rubber, urethane, or other materials for conveying acoustic energy to and from the body with minimal reflection or acoustic loss. Alternatively, the acoustic window 16 is an opening. The acoustic window 16 is the primary portion of the transducer assembly 12 for contact with the patient. Various temperature regulations apply to the acoustic window 16. Because of thermal conduction, heat generated by the transducer 15 and elsewhere within the housing 18 may result in an elevated temperature of the acoustic window 16.

The transducer housing 18 is Pebax, plastic, epoxy, metal, fiberglass or other material for housing the transducer 15. The transducer housing 18 is shaped for being hand held by a sonographer. Alternatively, the transducer housing 18 is shaped for insertion into a patient, such as shaped as a catheter, an endo-cavity probe, a transesophageal probe or an intra-operative probe. In one embodiment, the transducer housing 18 also includes active electronics, such as amplifiers, transistors, waveform generators, digital-to-analog converters and/or digital to optical converters. Active electronics also generate heat.

Waste heat generated by components within the transducer housing 18 is removed to preclude the surface temperature exceeding regulatory limits. Thermal conduction to the body and to the other components within the housing 18 transfers the heat away from the sources of heat, such as the transducer 15. Waste heat is transferred down the thermal gradient through the thermal plate 20. The thermal plate 20 is copper, aluminum, other metal or other material providing thermal conduction. The thermal plate 20 is positioned immediately adjacent to one or more sources of heat, such as along the sides of a transducer stack, or connects with other thermal conductors, such as a grounding plane. In one example, the thermal plate has a ten degree C. steady state temperature. More than one thermal plate 20 may be provided. In alternative embodiments, the thermal plate 20 is flexible, has other non-plate shapes or other steady state temperatures.

The heat exchanger 22 connects with or is formed as part of the thermal plate 20. The heat exchanger 22 is copper, aluminum, other metal or other thermally conductive material. The heat exchanger 22 has a large surface area connection with the thermal plate 22, but a smaller surface area may be provided. The heat exchanger 22 also includes one or more internal channels for thermal transfer to the coolant flowing through fluid path 24. Alternatively, the fluid path 24 is positioned adjacent to the heat exchanger 22 or to the thermal plate 20 without the heat exchanger 22. In one example, the heat exchanger 22 has an average four degree C. steady state temperature.

The fluid path 24 is a tube of Mylar, Pebax, PTFE, urethane, HDPE or other material that is compatible with the circulating coolant. The fluid path 24 encapsulates a coolant, such as Freon, Flourinert, ethylene glycol, propylene glycol, alcohol or any other liquid or gas that avoids freezing at the temperatures of use. Liquids with high specific heats and low viscosities are preferred. The fluid path 24 extends from the connector 26, through the cable 13, to the transducer housing 18, such as adjacent to the transducer 15. In the connector 26, the fluid path 24 extends adjacent to or into the thermally conductive shoe 32. The fluid path 24 is a closed loop.

The temperature difference between the warmer heat exchanger 22 and the cooler circulating coolant 24 causes heat to be transferred into the coolant, increasing its temperature from −2 degrees C. as the coolant enters the transducer housing 18 to 9 degrees C. as the coolant leaves the transducer housing 18. Since the circulating coolant in the cable is below the typical ambient air temperature of about 25 degrees C., heat is extracted from the atmosphere as the coolant travels from the connector 26 to the transducer housing 18, and back. In this example, this heat causes the coolant to increase in temperature by 2 degrees as the coolant travels each way between the transducer 15 and the connector 26. The resulting increase temperature of the coolant entering the transducer housing 18 decreases the amount of waste heat that can be removed from the heat generating components within the transducer housing 18.

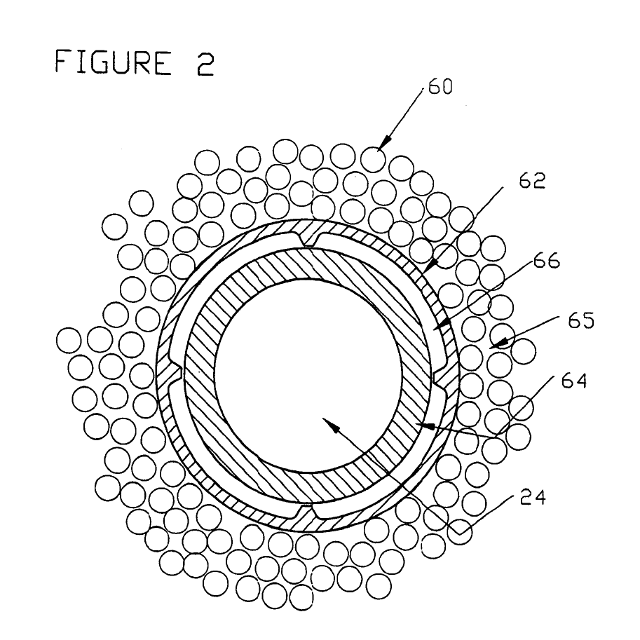

FIG. 2 shows one embodiment of the fluid path 24 along the transducer assembly cable to reduce heat transfer from the surroundings. The fluid path 24 is positioned to be surrounded by a plurality of coaxial conductors 60. The coaxial conductors 60 are used for conducting electric transmit pulses generated either in the imaging system 14 or within the connector 26. Receive signals from the transducer 15 are conducted to the connector 26 using the same conductors or using alternate conductors. The coaxial conductors 60 and associated air gaps 65 provide some thermal insulation. Further insulation is provided by a layered tube for the fluid path 24. An outer layer 62, such as extruded Mylar or PTFE, is around an inner layer 64, such as extruded PTFE or other material compatible with the coolant. The inner layer 64 and/or the outer layer 62 have ridges or separators to create and maintain a gap 66 between the layers 62 and 64. The gap 66 is filled with air, an insulator or other material to further reduce heat transfer.

Again with regards to FIG. 1, the purpose of pump 28 is to recirculate the coolant through the closed fluid path 24. The pump 28 includes an integrated electric motor. The pump 28 can use centrifugal, fixed displacement, diaphragm or other methods to move the fluid. The pump 28 is within the connector 26 of the transducer assembly 12. The pump 28 is separate from or integrated into the thermally conductive shoe 32. Electrical power is provided to the pump 28 from the ultrasound system 14, such as through electrical interconnects or contacts between the connector 26 and the ultrasound system 14. The pump 28 increases the pressure of the coolant to overcome frictional losses associated with moving coolant through the fluid path 24.

In an alternative embodiment, the pump 28 is within the ultrasound transducer assembly 12 and mechanically interconnects with a motor, located in the ultrasound system 14. A shaft, rotated by the motor, causes the pump 28 to operate. In one embodiment, the shaft includes a detachable linkage or coupler for connecting the pump shaft and the motor shaft together between the transducer assembly connector 26 and the connector of the ultrasound system 14. In another embodiment, the coupling is magnetic with no mechanical interface. The drive motor is in a location that can be conveniently powered by the ultrasound system 14. The amount of electrical power available in the ultrasound system 14 is greater than the amount that can be transferred to the connector 26 through normal interconnection methods. This may be useful for a refrigeration system located within the connector 26 as refrigeration systems consume relatively large amounts of power. There may also be RFI advantages to locating the drive motor within the imaging system 14 because of the practicality of implementing space consuming shielding or electrical filtering.

Again with reference to FIG. 1, the spring 34 is a single or multiple springs, capable of generating a normal force between the thermally conductive shoes 32 and 40. A lever arm or other devices for applying a normal force between the thermally conductive shoe 32 and the connector thermally conductive shoe 40 may additionally or alternatively be used. The normal force improves the efficiency of the thermal interconnection between the two conductive shoes, 32 and 40.

The thermally conductive shoe 32 is a plate, block, or other shaped material. Copper, gold plated copper, silver, aluminum, other metal or other thermally conductive material is used. The mating surface 30 of the thermally conductive shoe 32 is flat with a surface area such as ½ to 2 square inches. In other embodiments, the surface 30 is not flat, such as having fins for fitting into corresponding slots. The thermally conductive shoe 32 includes one or more fluid channels, such as a circuitous path of the fluid path 24. The channels within the thermally conducive shoe 32 are designed to maximize the efficiency of heat transfer from the warmer coolant 24 to the cooler thermally conductive shoe 32. The channels of the fluid path 24 are about 3 mm from each other, but greater or lesser separation with a single or multiple loops may be provided. The heated coolant 24 from the transducer housing 18 is circulated through the thermally conductive shoe 32 where the coolant temperature is reduced by 15 degrees because of heat transfer to the lower temperature thermally conductive shoe 40.

In the ultrasound system 14, the other thermally conductive shoe 40 is a same or different material, shape and construction as the thermally conductive shoe 32 of the transducer assembly 12. The system thermally conductive shoe 40 is a solid material operable to contact or mate with the solid thermally conductive shoe of the transducer assembly 12 when the connector 26 is connected with the ultrasound system 14. The conductive shoes 32,40, provide a thermal interconnection without fluid transfer with heat being transferred by conduction. Flat mating surfaces and/or a modest normal force generated by the spring 34 or other structure in the connector 26 or in the system 14 assure an efficient thermal path or connection. The temperature of the system thermally conductive shoe 40 is −10 degrees C., resulting in a 2 degree temperature difference between the mating shoes to transfer the waste heat into the system 14.

The system thermally conductive shoe 40 includes features for transferring heat to the refrigeration system in the ultrasound system 14. A refrigerant path 46 passes through or beside the system thermally conductive shoe 40. The refrigerant path 46 is a tube of copper, other metal or other compatible material that encapsulates the refrigerant. Freon 134a is an example of refrigerant that is present in both vapor and liquid states at different locations within the refrigerant path 46. The refrigerant path 46 extends from adjacent to or in the system evaporator thermally conductive shoe 40 through the compressor 48 to the condenser 50, through the orifice 44 back to the evaporator 40. The fluid path 46 is a closed loop, located within the imaging system 14 and separate from the fluid path 24 of the transducer assembly 12.

As the refrigerant in vapor form passes through the compressor 48, the refrigerant's temperature is increased to a temperature significantly above the temperature of the ambient air, by essentially adiabatic compression. This hot, high pressure vapor, then moves to the condenser 50 where significant heat is transferred to the internal surfaces of the condenser 50. As heat is extracted from the vapor, the vapor condenses to a liquid at nearly the same temperature. This heat liberated from the nearly isothermal phase change is called the latent heat of vaporization. When the high-pressure refrigerant exits the condenser 40, the refrigerant is mostly liquid. The temperature of the refrigerant is nearly the same as the temperature of the high-pressure vapor entering the condenser.

The high-pressure liquid travels to the orifice 44 at the entrance to the evaporator. The pressure of the flowing liquid decreases as it passes through the orifice 44 and enters the evaporator 44. The low-pressure liquid refrigerant flashes to vapor in the evaporator 40 and extracts the required latent heat of vaporization from the inner passages of the evaporator 40. Heat extracted from the evaporator 40 causes it to decrease in temperature. The resultant low pressure, low temperature gaseous refrigerant is then returned to the compressor 48 to repeat the continuous process.

In one embodiment, the orifice 44 size is adjustable so that it can be used to control the amount of refrigeration achieved. Small orifices are associated with high heat transfer rates. As the orifice size is increased, the refrigerant back-pressure in the condenser decreases. The resultant pressure increase across the compressor 48 is diminished. The resultant lower coolant temperature out of the compressor 48 and entering the condenser 50 reduces the heat transfer rate. If the orifice is opened completely, the energy used to operate the compressor ends up as heat, causing the evaporator 40 to actually increase in temperature. In alternative embodiments, the orifice 44 is positioned at a different location, such as integrated within the thermally conductive shoe 40 or spaced away from the thermally conductive shoe 40.

The heat exchanger 50 is a metal or other structure with the fluid path 46 adjacent to or within the structure operating as a liquid/air heat exchanger or condenser. Fins 52 are provided for transferring heat (e.g., 50 degrees C.) to the atmosphere. Heat is transferred from the system thermally conductive shoe 40 (evaporator) to the condenser 50 for dissipation into the ambient air by radiation or by forced convection. The energy to pump heat up the temperature gradient is supplied by the compressor 48. A small fan 54 is used to circulate the cool ambient air through the heat exchanger. In another embodiment, fans already used within the system to cool other components provide the air circulation.

The refrigeration system 49 maintains the thermally conductive shoe 40 at a temperature less than that of the ambient air. Consequently, a steeper temperature gradient is provided within the transducer assembly 12. Thus, more heat may be extracted from the transducer 15 and dissipated into the atmosphere. In this example, the thermal interface at the thermally conductive shoe 32 is at −10 degrees C. Without a refrigeration system, the thermally conductive shoe 40 would be at a minimum temperature of 25 degrees C., the ambient air temperature.

There are several methods for designing active cooling or refrigeration systems. In general, refrigeration transfers heat up a thermal gradient. This is counter to the normal situations where heat flows from a higher temperature region to a lower temperature region through conduction, radiation, or convection. Electrical or other forms of energy must be supplied to the refrigeration active cooling device 49. Although refrigeration uses external forms of energy, when applied to the active cooling transducer assembly 15, refrigeration allows extraction of significantly greater quantities of heat than would be otherwise possible.

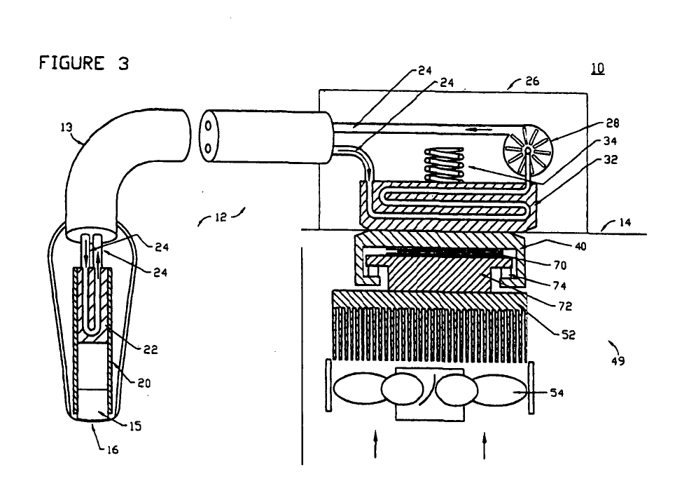

FIG. 3 shows an alternative embodiment of the refrigeration system 49. The refrigeration system 49 includes the fan 54, fins 52, adapter 72, springs 74 and a thermal electric device 70. Additional, different or fewer components may be provides, such as not providing the fan 54, fins 52, adapter 72 and/or springs 74. The transducer 12 is functionally identical to that described above in FIG. 1.

The thermal electric device 70 is a thermo-electric cooler. Thermo-electric cooling devices exploit the Peltier-Effect to cause heat to flow between fused, dissimilar metal surfaces when subjected to a DC current. In one embodiment, the thermal electric cooler 70 is 1.75 inches by 1.56 inches device and about 0.093 inches thick. Such a thermal electric cooler 70 may be able to move 50 watts of power, in the form of heat, against a 20-degree temperature gradient using approximately 100 watts of electrical power. A Marlow XLT2385 is an example of a commercially available thermo electric cooler. 40 watts of heat may be transferred from a structure that is 30 degrees C. to an adjacent structure that is 50 degrees C. by using a DC current of 9 amps and a potential difference of 5.5 Volts. Thus, 50 watts enters the cold face and 90 watts exits the hot face. Other thermal electric devices 70 disclosed in U.S. Pat. No.______ (application Ser. No. 10/183,302), the disclosure of which is incorporated herein by reference, may be used. More or less efficient devices with a greater or lesser amount of thermal capacity may be provided for a greater or lesser gradient.

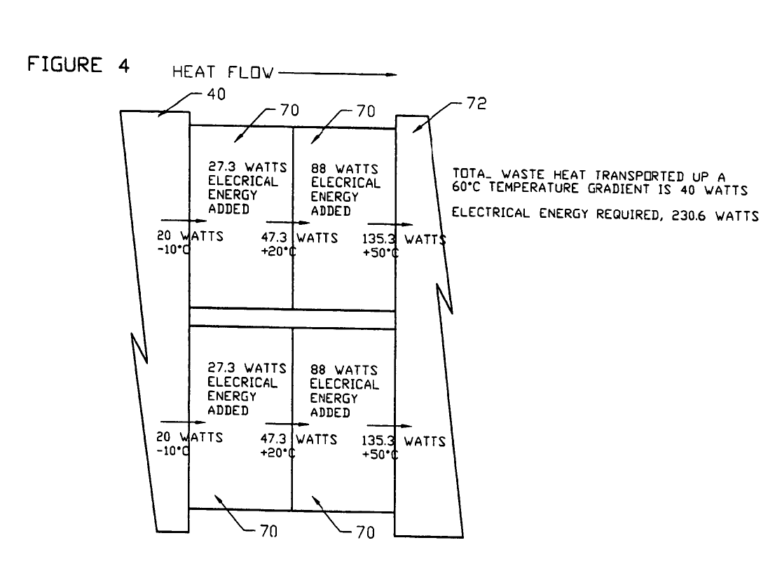

To pump heat across greater temperature rises, multiple thermal electric devices 70 are cascaded in series. To increase the amount of heat pumped across a given temperature rise, multiple thermal electric devices 70 are positioned in parallel. Additional thermal electric devices 70 use additional energy. For example in FIG. 3, 40 watts of heat is pumped from the system thermally conductive shoe 40 (−10 degrees C.) to the extrusion adaptor 72 (50 degrees C.) using four thermal electric devices 70, two series stacks of 2 are paralleled in this example. FIG. 4 is a thermal diagram of this configuration. In this example, a total of 230.6 watts electrical power is required to pump 40 watts of heat from −10 degrees C. to +50 degrees C. Thus, a total of 270.6 watts is dissipated to the 25 degree C. atmosphere from the metal finned structure 52 at 48 degrees C. For this example, a fan/heat exchanger assembly (52 and 54) with a thermal resistance of about 0.185 degrees C./Watt is used.

As compared to the components comprising the vapor/liquid refrigeration system illustrated in FIG. 1, the thermal electric devices 70 are relatively small, and are, in general, less efficient. Refrigeration systems based on thermal electric devices offer several packaging advantages because of their size. Electrical power to operate the accessory thermoelectric coolers could be extracted from the imaging system, supplied by a separate source, or powered by a battery or a fuel cell located either on the imaging system or in a remote location. Thermo-electric coolers, because of their compact sizes, may allow an enhanced cooling system to be mounted on an existing imaging system as an accessory.

Again with reference to FIG. 3, the adaptor 72 is aluminum, copper, other metal or other thermally conductive material sized and shaped to sandwich the thermal electric devices 70 against the system thermally conductive shoe 40. The shoe 40 and adaptor 72 also connect through one or more springs 74, compressed rubber spacers or other material operable to dispose the adapter 72 against the thermal electric devices 70. Alternatively, the metal finned structure 52 presses the thermal electric devices 70 against the shoe 40, without use of an adapter 72.

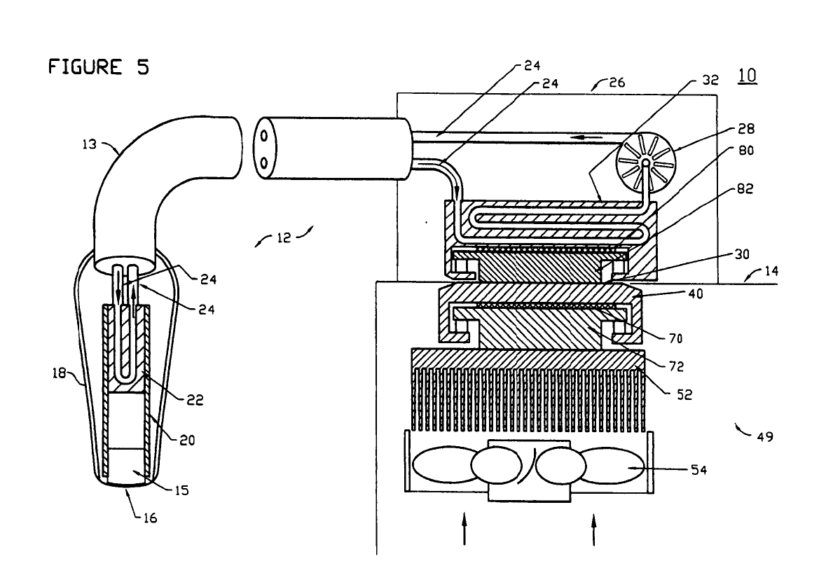

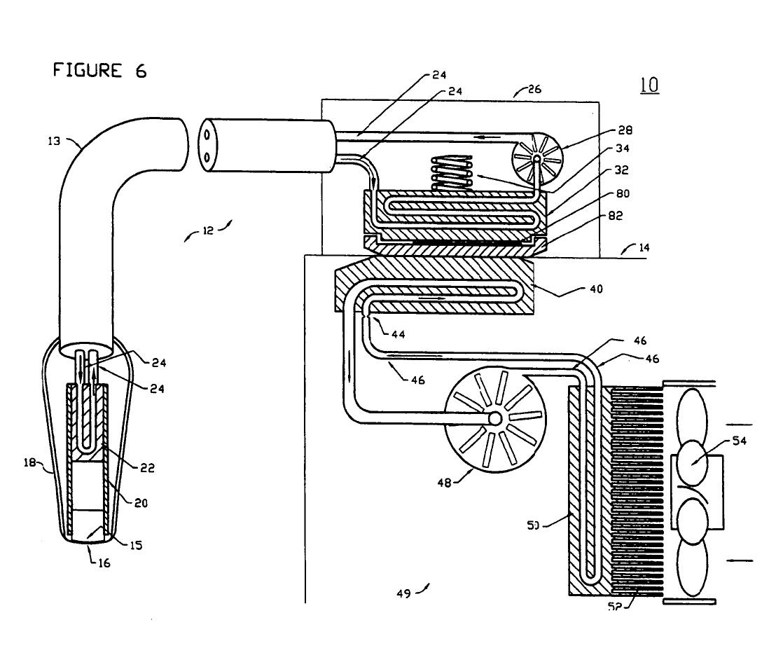

FIGS. 5 and 6 show two other embodiments providing an additional active cooling device 80 within the ultrasound transducer assembly 12. FIG. 5 shows the additional active cooling device 80 within the transducer connector 26 and with a thermal electric device 70 in the ultrasound system 14. FIG. 6 shows the additional active cooling device 80 within the connector 26 in addition to the vapor/liquid refrigeration system 49 located in the ultrasound system 14. The additional active cooling device 80 is a single or multiple thermal electric coolers positioned adjacent to the thermally conductive shoe 32 in the connector 26 of the ultrasound assembly 12. The additional thermal electric active device 80 may use 50+ watts or other amount of electrical energy to operate. The additional active cooling device 80 is powered through one or more interconnections or electrical contacts with the ultrasound system 14 operable for the wattage used by the additional active cooling device 80. It is also possible that a small refrigeration system (vapor/gas) located within the connector 26 may be powered by a remotely located motor in the ultrasound system 14 for providing further active cooling in the transducer assembly connector 26.

With reference to FIG. 5, the adapter 82 mates with the system thermally conductive shoe 40, such as providing a flat surface. The adapter 82 is copper, gold plated copper or other thermally conductive material. Springs 74 or 34 press the additional active cooling device 80 between the adapter 82 and the shoe 32.

The additional active cooling device 80 may result in the mating surfaces of the system thermally conductive shoe 40 and the adapter 82 having a temperature closer to ambient, such as at about 20-25 degrees C., even though the coolant in fluid path 24 is at a far lower temperature. The thermally conductive shoe 40 and adapter 82 are less likely to condense moisture out of the atmosphere or freeze together. For example, the additional active cooling device 80 provides a temperature gradient of about 33 degrees C. The adapter 82 is at 25 degrees C. The system thermally conductive shoe 40 is at about 23 degrees C. For FIG. 5, the thermoelectric cooling device 70 in the ultrasound system 14 provides a 33 degree C. temperature rise so that the adapter 72 is at 56 degrees C. The heat exchanger fins 52 are at 53 degrees C. with air being heated from the ambient temperature of 25 decrees C. to 45 degrees C.

For FIG. 6, the refrigerant, in liquid form, entering the orifice 44 is at 50 degrees C. and 148 psi. As it passes through the orifice 44, the pressure decreases to 20 psi, and the temperature decreases to −20 degrees C. As the refrigerant travels through the evaporator shoe 40, the refrigerant changes from liquid to a vapor. The gaseous refrigerant exiting the shoe 40 is about −15 degrees C. at 20 psi. As the pressure is increased in the compressor 48 from 20 psi to 148 psi, the temperature increases to 50 degrees. After the refrigerant condenses to liquid within the condenser 50, the refrigerant temperature is still 50 degrees C. The cycle thus repeats. Ambient air, forced into the fins 52 increases from 25 degrees C. to 40 degrees C., before it is exhausted.

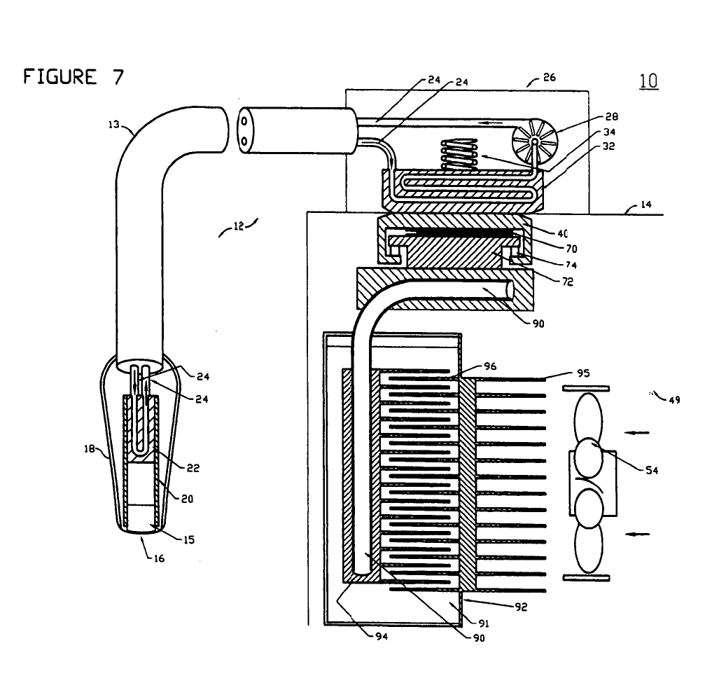

FIG. 7 shows an embodiment where the refrigeration system 49 in the ultrasound system 14 also includes a heat pipe 90 and/or a thermal storage tank 92. The heat removal rate may exceed the rate using just the fins 95 and fan 54.

The heat pipe 90 is an enclosed structure of aluminum, copper or other material that contains a heat transfer medium in both vapor and liquid form. The heat pipe 90 is about ¼ inch in diameter, but may be larger or smaller. The heat transfer medium is water, alcohol, acetone, Freon or other substance. Materials with high latent heats of vaporizations are preferred to maximize the performance capabilities of the heat pipe. Heat transferred into the evaporator section from the adapter 72 is absorbed by the liquid, causing it to change to a vapor. As the vapor is generated, the vapor travels towards the slightly cooler condenser section where the vapor liquefies after depositing the heat of vaporization on the inside walls of the heat pipe 90. Because evaporation and condensation occur at essentially the same temperature, the heat pipe 90 has a very high effective thermal conductivity when compared to an equivalent solid material, such as a metal. Relatively small heat pipes 90 can transfer large amounts of heat with very little temperature gradient. Condensed liquid is returned to the evaporator section using gravity or using some structure or mesh that exploits capillary action behavior of liquids.

The thermal storage tank 92 is a metal or other material structure for housing a phase change medium 91. Using the thermal storage tank 92, the heat removal rate from the transducer 15 can exceed the ability of the system to dissipate heat into the atmosphere. The waste heat is not dissipated into the atmosphere as quickly as it is generated by the transducer 15 or by the active electronics located either in the transducer housing 18 or the connector 26. The waste heat, not otherwise dissipated, is stored in the phase change medium 91 for dissipation at a later time. Cetyl alcohol is an example of such a medium with a fusion temperature of about 50 degrees C. and a relatively high heat of fusion. Thus, the system does not operate continuously at steady state. This particular system is practical for diagnostic ultrasound equipment since diagnostic procedures are generally not done on a continuous basis.

The condenser section of the heat pipe 90 is thermally common to the thermally conductive liquefier structure 94, located within the thermal storage tank 92. Heat, transferred to the medium 91 by the liquefier 94, causes an amount of the medium to be converted from a solid to a liquid, consistent with the heat of fusion for that material. Also encapsulated in the thermal storage tank 92 is an air/liquid heat exchanger (solidifier) 96. The solidifier 96 and fins 95 are copper, aluminum or other thermally conductive material. Heat is transferred from the warmer liquid medium 91 to the cooler ambient air by the solidifier 96 and the fins 95. Removal of this heat of fusion from the liquid causes the medium to solidify. The heat transfer from the solidifier to the ambient air is enhanced by the presence of the fan 54. Close proximity of the heat transfer surfaces of the liquefier 94 with corresponding surfaces of the solidifier 96 minimizes or eliminates the need for a pump to physically circulate liquid medium 91 within the storage tank 92. Alternatively, a pump in the thermal storage tank 92 transfers the liquid medium 93 from the proximity of the liquefier fins 94 to the solidifier 96.

In this example, the active cooling hardware 20, 22, within the transducer housing 18 extracts heat at the rate of 40 watts from the transducer 15. Because of the thermal electric coolers 70, a total of 270.6 watts is either stored in the medium 91 or dissipated to the atmosphere by the fins 95. If the fan 54 and finned radiator 96 are only capable of dissipating 75 watts to the atmosphere, then 0.195=kilowatt-hours of energy are stored if the transducer 15 is used at full power for an entire hour. The rate of dissipation of heat from the solidifier 96 to the ambient air can be increased by increasing the air velocity using a more aggressive fan 54, or by increasing the surface area of the solid/air heat transfer surfaces. The benefit of increasing the rate of heat transfer is the reduction of the amount of energy that must applied to the thermoelectric devices.

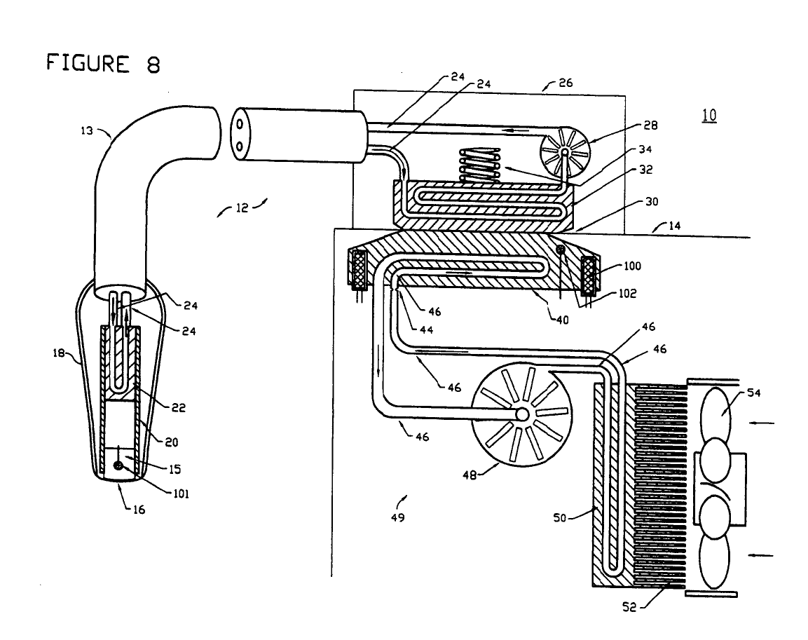

With reference to FIG. 8, the refrigeration system 49 can be controlled to directly regulate the temperature of the thermally conductive shoe 40, or to indirectly regulate the temperature of the acoustic window 16. A programmable controller, such as a micro-controller, field programmable gate array, analog circuit, digital circuit or other controller, controls operation of the orifice 44, the compressor 48, fan 54, and/or the pump 28, based on temperatures measured by sensor 102 located within the thermally conductive shoe 40. The controller can be physically located either in the transducer assembly 12 or in the imaging system 14. In addition or alternatively, the temperature sensors 102, such as thermocouples, thermistors or RDT's (Resistance Temperature Detector), are positioned within or in close proximity to the transducer 15, the heat exchanger 22, the transducer assembly shoe 32, the system shoe 40 and/or other locations, such as within the fluid paths 24 and/or 46.

The amount of heat generated in the transducer 15, the transducer supporting electronics, located within the housing 18, and/or the active electronics located in the connector 26 depends on the design of these components and on the way in which they are used to obtain diagnostic information from a patient. Reliable heat removal from the components is used to assure that transducer surface temperatures do not exceed regulatory limits and that the electronics components are not damaged by the excessive temperatures.

Active cooling systems, especially refrigeration active cooling systems, consume considerable amounts of energy during their operation. Several of the components of these systems are maintained at temperatures below the ambient air temperature. These low temperatures can cause condensation of atmospheric humidity, and/or cause the formation of frost. The controller operating the cooling system components can be used to avoid or limit condensation or frost. The transducer waste heat removal rate can be controlled in several ways. With thermo-electric cooling devices 70, 80, the heat removal rate is determined by the amount of electrical current passed through the device. By reversing the current, thermo-electric devices will transport heat in the opposite direction, providing a heating effect. For the vapor/liquid refrigeration approach, the heat removal rate can be controlled by adjustment of the expansion valve (orifice 44), by cycling the compressor 48 on and off, or by controlling the airflow through the condenser.

Since the imaging ultrasound system 14 controls the operation of the transducer assembly 12, the amount of waste heat that will be generated in the transducer components may be estimated, based on previous experimental tests. In addition to temperature sensing described above, the controller can control the waste heat removal rate for the various operational modes based on algorithms as a function of the operation of the transducer assembly 12. For example, a greater amount of waste heat removal is provided for continuous wave imaging than for triggered contrast agent imaging.

With reference to FIG. 8, in another or additional embodiment, a temperature sensor(s) 102, located in the thermally conductive shoe 40 or a temperature sensor(s) 101 located in the transducer 15, can be used to generate information used to control the cooling system. The heat removal rate is increased for detected temperatures greater than preset values. The heat removal rate is decreased for temperatures below the same or other preset values. The amount of increase or decrease may be based on other thresholds. In one embodiment, the heat removal system would be operated at the minimum level to keep the temperatures within predetermined limits.

In another embodiment, the heat removal control system is optimized to control the temperature of the thermally conductive shoes 32 and 40 when the transducer is not being used. During normal operation, the shoes 32 and 40 operate at temperatures significantly below the ambient air temperature; this can cause condensation of moisture from the atmosphere. If the moisture intrudes into the delicate electronics in either the connector 26 or the imaging system 14, reliability problems can result. In extreme cases, the moisture formed on the conductive shoes 32 and/or 40 can freeze; this would preclude the removal of the transducer assembly 12 from the imaging system 14, or preclude the installation of the transducer to the imaging system.

Both the thermo electric cooler and the vapor/gas refrigeration systems 49 may be operated to generate heat in the shoes 32, 40, illustrated in FIG. 3. The thermal electric cooler device 70 powered by a DC current with a polarity reversed from the normal operational modality. Operated in this manner, the thermal electric device would actually become a heater. In the embodiment using a liquid/vapor refrigeration system shown in FIG. 8, the compressor 48 can be operated in a reversed direction, causing the thermally conductive shoe 40 to increase in temperature. An alternative way of heating the thermally conductive shoe 40 would be to open the orifice 44, as discussed above.

FIG. 8 shows one embodiment using heaters 100 other than or in addition to the thermal electric cooling devices 70, 80 or the compressor 48. The heater 100 comprises an electric cartridge heater. The heater 100 and none, one or more temperature sensors 102 are located in or adjacent to the system thermally conductive shoe 40 and/or the transducer assembly thermally conductive shoe 32. Two heaters 100 are shown, but one or three or more may be provided. A closed loop temperature controller is used to determine the temperature of the shoe 40 and make decisions as to how much current to run through the heaters 100 to maintain a pre-programmed temperature level, such as atmospheric temperature. This controller also monitors the imaging system operational requirements, and would override a pre-programmed temperature level, and/or operate the compressor 48 to provide cooling.

A method is provided for cooling an ultrasound transducer. The method used one of the embodiments above or a different embodiment. Active cooling is performed within an ultrasound system by refrigeration. For example, ultrasound systems are cart mounted imaging devices for medical diagnostic use. Beamformers and image processors in the ultrasound system generate diagnostic images or information. Refrigeration devices are also positioned within the ultrasound system, such as in a same cart, housing or frame.

Transducer assemblies are releasably detachable with the ultrasound system to scan a patient with ultrasound energies. During operation, the transducer and any integrated active electronics generate heat. The heat is conducted or transferred from the ultrasound transducer. In response to the refrigeration within the ultrasound system, the heat is conducted to the ultrasound system without a fluid connection between the ultrasound transducer and the ultrasound system. Instead of fluid transfer, heat is conducted from the transducer assembly to the ultrasound system through the respective connectors. A thermal block in the ultrasound transducer assembly connector is mated with a thermal block in the ultrasound system connector. Heat is conducted through the thermal blocks.

In one embodiment, the refrigeration system 49 is in an adaptor positionable within an imaging system or between the connector 26 and the imaging system 14. The adaptor is used to retrofit existing systems for active cooling. The connector 26 of the transducer assembly 12 includes the shoe 32 for conductive mating with a shoe 40 in the adaptor.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for cooling an ultrasound transducer, the system comprising:
an ultrasound transducer assembly;
an ultrasound system, the ultrasound transducer assembly operable to releasably connect with the ultrasound system;
a refrigeration device within the ultrasound system; and
a connector operable to thermally conduct between the refrigeration device and the ultrasound transducer assembly free of fluid transfer.

2. The system of claim 1 wherein the refrigeration device comprises a compressor with a heat exchanger.

3. The system of claim 1 wherein the refrigeration device comprises a thermal electric cooler.

4. The system of claim 1 wherein the refrigeration device comprises a heat pipe, a thermal storage tank or combinations thereof.

5. The system of claim 1 wherein the connector comprises a metallic shoe;

6. The system of claim 5 wherein the metallic shoe comprises a fluid channel.

7. The system of claim 1 wherein the connector comprises a first solid material in the ultrasound transducer assembly and a second solid material in the ultrasound system, the first and second solid materials operable to contact each other through application of normal force while the transducer assembly is connected with the ultrasound system.

8. The system of claim 1 wherein the ultrasound transducer assembly further comprises:
a first fluid path extending from the connector to a transducer array housing; and
a pump operable to circulate fluid within the first fluid path.

9. The system of claim 8 wherein the refrigeration device comprises a second fluid path extending to the connector.

10. The system of claim 8 wherein the ultrasound transducer assembly comprises a plurality of coaxial cables extending between the connector and the transducer array housing, the plurality of coaxial cables positioned around the first fluid path, the first fluid path having inner and outer tubes with a gap between the inner and outer tubes.

11. The system of claim 8 wherein the pump is within the ultrasound transducer assembly and connects with a motor in the ultrasound system through the connector.

12. The system of claim 1 further comprising an air heat exchanger connected with the refrigeration device in the ultrasound system.

13. The system of claim 1 further comprising an additional refrigeration device within the ultrasound transducer assembly.

14. The system of claim 1 further comprising:
a heater adjacent the connector.

15. The system of claim 1 further comprising:
a controller operable to regulate temperature in response to a temperature sensor or use of a transducer array of the ultrasound transducer assembly.

16. A system for cooling an ultrasound transducer, the system comprising:
an ultrasound transducer assembly having a first fluid path extending from adjacent a transducer array to a first thermally conductive shoe in a first connector; and
an ultrasound system having a refrigeration device, having a second connector operable to connect with the first connector and having a second thermally conductive shoe in the second connector, the second thermally conductive shoe positioned to contact the first thermally conductive shoe if the ultrasound transducer assembly is connected with the ultrasound system, the refrigeration device thermally connected with the second thermally conductive shoe.

17. The system of claim 16 wherein the first and second connectors are free of fluid connection.

18. The system of claim 16 wherein the refrigeration device comprises: a compressor with a heat exchanger, a thermal electric cooler or both.

19. The system of claim 16 wherein the first fluid path extends into the first thermally conductive shoe and a second fluid path separate from the first fluid path extends into the second thermally conductive shoe.

20. A method for cooling an ultrasound transducer, the method comprising:
actively cooling within an ultrasound system; and
conducting heat from the ultrasound transducer in response to the active cooling within the ultrasound system free of fluid connection between the ultrasound transducer and the ultrasound system.

21. The method of claim 20 wherein conducting heat comprises conducting the heat through a first thermal block in an ultrasound transducer assembly connector mated with a second thermal block in the ultrasound system.

22. A method for cooling an ultrasound transducer, the method comprising:
generating waste heat in a transducer;
transferring the waste heat to an imaging system with conduction;
rejecting the waste heat into the atmosphere within the imaging system.

23. The method of claim 22 further comprising:
measuring a temperature adjacent the transducer; and
regulating the transferring and rejecting as a function of the temperature.

24. The method of claim 22 wherein transferring and rejecting comprise preventing a temperature from exceeding a set-point.

25. The method of claim 22 further comprising:
controlling the transferring and rejecting as a function of transducer operation.

26. The method of claim 22 further comprising:
operating thermoelectric coolers, resistive heaters or combinations thereof; and
limiting moisture formation as a function of the operating.

27. The method of claim 22 wherein rejecting comprises rejecting with a refrigeration system in the imaging system;
further comprising controlling operation of the refrigeration system with a controller in a transducer assembly for the transducer.

28. The method of claim 22 further comprising:
maintaining an interface between a transducer assembly for the transducer and the imaging system substantially at ambient temperature when the transducer is not in use.

29. A retrofit system for cooling an ultrasound transducer, the system comprising:
a refrigeration system in an adaptor;
an adaptor connector on the adaptor for connection with a transducer assembly connector; and
a solid phase thermal conductor within the adaptor connector, the solid phase thermal conductor connected with the refrigeration system.
